(19) United States
(12) Patent Application Publication
(10) Pub. No.: US 2005/0177179 A1
(43) Pub. Date: Aug. 11, 2005

(54) Surgical cable system

(76) Inventors: Bret O. Baynham, Jupiter, FL (US); G. Clay Baynham, Jupiter, FL (US); Matthew G. Baynham, Jupiter, FL (US); David R. Campbell, Jupiter, FL (US)

Correspondence Address: MCHALE & SLAVIN, P.A., 2855 PGA BLVD, PALM BEACH GARDENS, FL, 33410, US

(21) Appl. No.: 10/776,728

(22) Filed: Feb. 10, 2004

(51) Int. Cl. A61B 17/10

(52) U.S. Cl. 606/151; 606/144; 606/142

(57) ABSTRACT

A surgical cable system for fixing bones in a spatial relationship is composed of a multistrand cable having improved flexibility with a fastener on one end and a free end. The fastener has an aperture through which the free end of the cable is inserted after being looped around the bones. The fastener has a stop in the aperture to crimp the cable to permanently fix the loop in the desired position. The tension on the cable is set using a provisional fastener and a tensioner instrument. The provisional fastener serves to separate the permanent fastener from the tensioner and align the cable to reduce friction and slack for more precise control of the tension and the size of the loop. The tensioner has a fixed length shaft providing small precise adjustments in the cable.

### BACKGROUND OF THE INVENTION

1. Field of the Invention

This invention relates to surgical apparatus, particularly an integrated cable, crimping and tensioning system.

2. Description of the Prior Art

Surgical cable is used in reconstructive spine surgery, such as fusion and spine trauma surgery, total hip replacement, fracture fixation, surgical closures, and the like. Cable and wire may be used to encircle broken bones to hold them together for healing. The cable may be monofilament or multifilament.

In general, surgical cable and wire require a length of cable to be applied about the skeletal member, a device or tensioner to apply tension to the cable to hold the skeletal member in the desired position, and a crimp or device to lock the cable in the preferred position. One conventional cable employs a titanium cable and a double lumen crimp. The crimp is a short rod with the double lumen passing longitudinally therethrough. The cable is passed through one lumen of the crimp, around the skeletal repair site and back through the other lumen. A tensioner is used to set the cable in place and a crimp is placed perpendicularly in the cable between the ends of the double lumen crimp. See U.S. Pat. No. 5,116,340 to Songer et al. Songer also discloses a tensioner device like a pair of pliers having opposed jaws for deforming the crimp on the cable. One of the opposing handles has a winding reel for creating the desired tension in the cable before the crimp is set. The cable may have an eyelet on one end through which the other end passes so that the deformed crimp is stopped by the larger eyelet.

A problem area for cable is the relative stiffness of the metal wires or strands and the relatively large diameter. These aspects detract from the ability to thread the cable through small skeletal openings and making sharp changes in direction. U.S. Pat. No. 5,997,542 to Burke addresses this problem and discloses another cable with one portion being more flexible and of smaller diameter than the rest of the cable. The smaller portion is used as a lead to thread the cable about the bones for placement. Burke also teaches an enlarged end on the cable by swaging or crimping the end of the cable in a blind bore of an enlargement.

Blackman et al, U.S. Pat. No. 6,146,386, discloses a surgical cable system with a tensioner operated by hand grips and ratchet. The cable is looped about bone anchors and tension is applied to displace the bones. Once the bones are properly located, the bone anchors fix the bones in place and the cable is removed from the body.

Wagner et al, U.S. Pat. No. 6,391,030, discloses a cable system with a cable, a tensioner and a connector for securing the cable in place about skeletal bones. The connector serves as a crimp to fix the size of the cable loop. The connector is separate from the cable and in one modification, the end of the cable has an enlargement to prevent disconnection. The connector has a pin that is rotatable to allow passage of the cable in one position and to secure the cable within the connector in another position. The pin is deformed upon locking the cable to prevent dislodgement.

Ferree, U.S. Pat. No. 6,514,255, discloses a cable system having a body which is movably mounted on a spinal rod and connected to a looped cable that extends around a vertebrae The body is moved to the preferred location on the rod and the cable loop is tightened to fix the body to the spinal rod and apply pressure to the hold the bones and body in place.

These prior art systems all apply the tensioner directly to the end of the cable and crimp which induces a certain amount of slack in the fixed cable. Usually, this is caused by the space needed to position the working end of the tensioner immediately adjacent the tissue and/or the several manipulative steps needed to crimp the cable and/or the stiffness of the cable in transitioning from the radius of the bone and into the crimp. More than one setting of the crimp is sometimes necessary to arrive at the preferred fixing of the cable tension.

### SUMMARY OF THE PRESENT INVENTION

Accordingly, it is an objective of this invention to provide a surgical cable system in which the first fixation of the permanent crimp is the final fixation at the proper cable tension and loop size.

It is another objective of the invention to provide a more flexible cable by increasing the number of filaments and decreasing filament size.

It is yet another objective of this invention to provide a permanent fastener with a low profile and passageway wherein the cable does not change direction, significantly, and reduces friction.

It is a further objective of this invention to provide a provisional clamp to remove the working end of the tensioner from the location of the permanent clamp during tensioning of the cable. The provisional fastener provides for one-way travel of the cable to maintain cable tension during adjustment of tension and relieves tension from the permanent clamp until that clamp is set.

It is another objective of this invention to provide a tensioner instrument for manually setting the tension on the cable. The instrument provides for one way movement of the cable that is precisely controlled and provides increased range of cable translation per cycle.

It is a still further objective of this invention to teach a method of placing a crimped surgical cable with a minimum number of adjustments. The components of the invention operate thusly; one end of the surgical cable is fixed to a permanent clamp and the free end of the cable is threaded around the skeletal processes to be held in place; the free end of the cable is then inserted through the permanent clamp; the provisional clamp is placed on the free end of the cable extending from the permanent fastener; the end of the cable extends through the provisional clamp longitudinally; the tensioner is placed on the provisional fastener and grips the free end of the cable; the hand grips of the tensioner are moved together to pull the free end of the cable to tighten the loop about the skeletal processes; the provisional fastener is constructed to allow the cable to move through the provisional fastener in one direction, only; as the hand grips are squeezed, the diameter of the loop is reduced and the proper tension is established in the cable; when the doctor or operator is satisfied with the placement, the tensioner is removed from the provisional fastener; the button on the side of the permanent fastener is moved to engage the cable in a non-slip crimp within the permanent clamp, the tension in the provisional clamp is then released so that the provisional fastener is slid off the free end of the cable; the excess cable is then cut. In this manner, the tension in the cable is maintained by both the provisional fastener and the tensioner with the permanent fastener and stop being free of tension until after the crimp is set.

### SHORT DESCRIPTION OF THE DRAWINGS

FIG. 1 is a side view of the permanent clamp and surgical cable of this invention;

FIG. 2 is an exploded perspective of the permanent clamp;

FIG. 3 is a cross section of the permanent clamp;

FIG. 4 a perspective of the tensioner of this invention;

FIG. 5 is a front view of the tensioner of this invention;

FIG. 6 is a longitudinal cross section of the tensioner;

FIG. 7 is a partial cross section of the tensioner of FIG. 5; and

FIG. 8 is a cross section of the provisional clamp of this invention.

### DETAILED DESCRIPTION OF THE INVENTION

A number of embodiments of the present invention have been described. Nevertheless, it will be understood that various modifications may be made without departing from the spirit and scope of the invention. Accordingly, it is to be understood that the invention is not to be limited by the specific illustrated embodiment but only by the scope of the appended claims.

The integrated cable system is composed of a surgical cable 11, a tensioner 12, a provisional clamp 13 and a permanent clamp 14. The cable may be a monofilament steel wire or a multifilament cable. The preferred cable has approximately 133 filaments of titanium alloy and a superior flexibility.

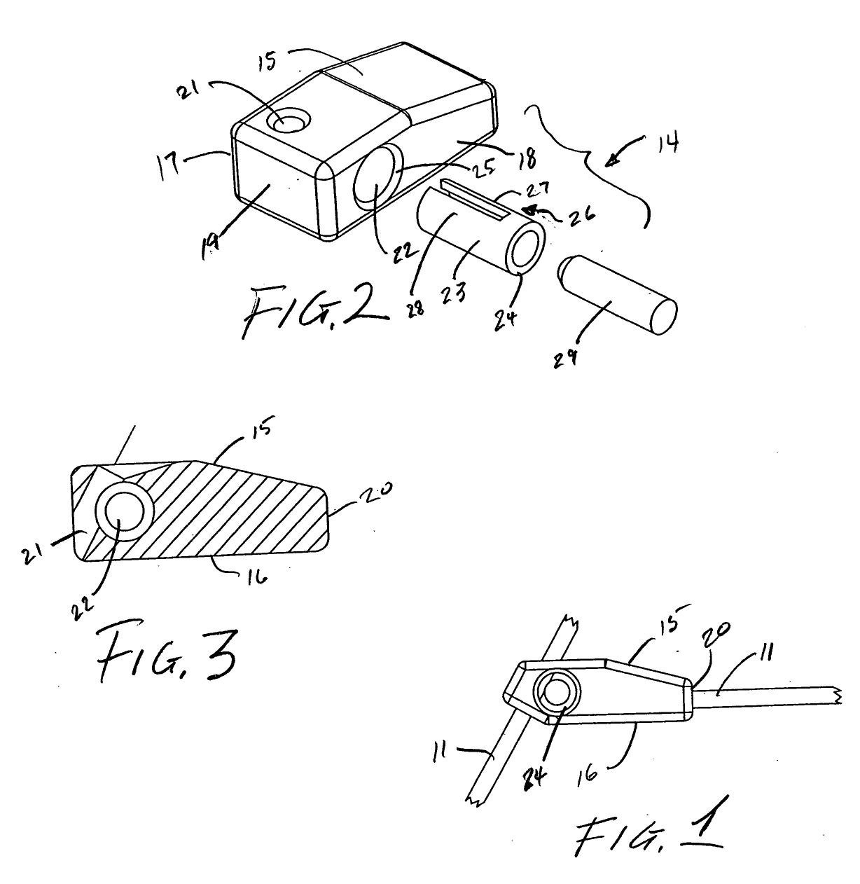

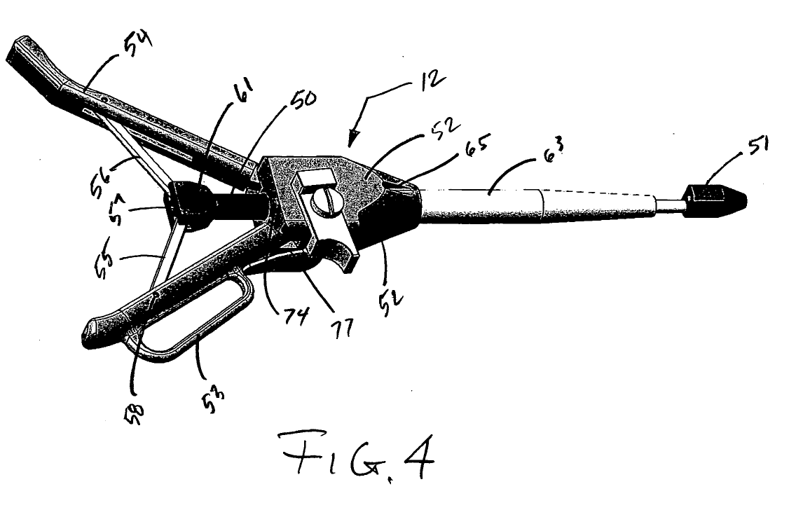

One end of the cable 11 is affixed to the permanent clamp 14, shown in FIGS. 1-3. Depending on the materials used, the clamp may be swaged onto the cable or soldered or welded or otherwise suitably joined. The permanent clamp has a rectangular body of low profile with a top wall 15, a bottom wall 16, opposite side walls 17, 18 connecting the top and bottom walls, and opposite front wall 19 and rear wall 20 connecting the top and bottom walls and perpendicularly connected to the side walls. The cable exits the rear wall 20 in line with the longitudinal axis of the clamp.

Near the front wall 19, a cable bore 21 extends through the permanent clamp from the bottom wall 16 through the top wall 5. The axis of the bore, as illustrated, is at an angle with the front wall 19. The angle is oriented to lessen the change in direction of the cable as it transits the permanent clamp. After the cable 11 has encircled the desired bones or bone fragments, the lead end of the cable is passed through the cable bore 21 and extends above the top wall. The cable bore 21 is of a diameter closely approximating the diameter of the cable 11. A lateral bore 22 passes through the side walls 17 and 18 tangentially intersecting cable bore 21 within the permanent clamp. Within the lateral bore 21 is a clamp mandrel 23. The mandrel 23 is movable from an open position to a clamping position which deforms the cable and/or obstructs cable bore and prevents retrograde movement of the cable 11 out of the permanent clamp 14. The end 24 of the mandrel is exposed in the open end 25 of the lateral bore and may be moved by either a push-pull motion along the axis of the mandrel or a rotational move about the axis. In the preferred embodiment, the mandrel 23 is composed of a split tube 26. The split tube 26 has semi-circular legs 27, 28 which extend across the tangential opening in the cable bore. The space between the semi-circular legs provides resilience in the split tube and allows the cable to pass through the cable bore during tensioning. The clamp is applied when the stop 29 is pushed into the split tube 26 causing the semi-circular legs to expand and wedge the cable 11 against the cable bore 21.

In operation, the cable is looped about the bones and through the permanent clamp. The cable and permanent clamp may be pre-assembled with the free end of the cable inserted through the clamp forming an adjustable loop. The leading end of the cable is threaded through the provisional clamp 13 and into the tensioner 12. The provisional clamp 13, shown in FIG. 8, has an inner tubular core 32 with a through bore 33 extending from the distal end 30 to the proximal end 31. The distal end 30 contacts the permanent clamp 14, about the cable bore 21, and the proximal end 31 contacts the tensioner 12. The proximal end of the core is formed as a disk 39. Between the distal and proximal ends of the core, there is an area 34 of reduced diameter having a proximal annular ledge 35. The inner core 32 has a transverse slit 36 oriented at an angle to the through bore with the lower end 37 of the slit intersecting the through bore 33. Within the slit 36 is a roller bearing 38 movable from one end of the slit to the other. When the roller bearing 38 is at the lower end 37 of the slit 36 it obstructs the through bore 33.

An outer sleeve 40 surrounds the core 32 and is telescopically movable along the axis of the inner core. Near the rearward end, an external flange 45 projects outwardly. Near the forward end of the sleeve there is an internal shoulder 41. Resting on the shoulder 41 is a ring 42. Between the ring 42 and the ledge 35 is a coil spring 49 encircling the reduced diameter area of the core. The ring 42 is in contact with the opposite ends of the roller bearing 38 thereby biasing the bearing toward the lower end of the slit 36 which permits movement of the cable from the permanent clamp toward the tensioner but prevents retrograde movement. Upon movement of the cable toward the tensioner 12, the ring will compress the spring slightly and upon release of the tension the roller bearing 38 is spring biased to wedge the cable in the through bore 33.

To release the cable in the provisional clamp, the disk 39 and flange 45 are used to telescope the inner core and outer sleeve into a compact position. This moves a portion of the slit 36 below the ring 42 allowing the roller bearing freedom to move away from the cable an out of the through bore.

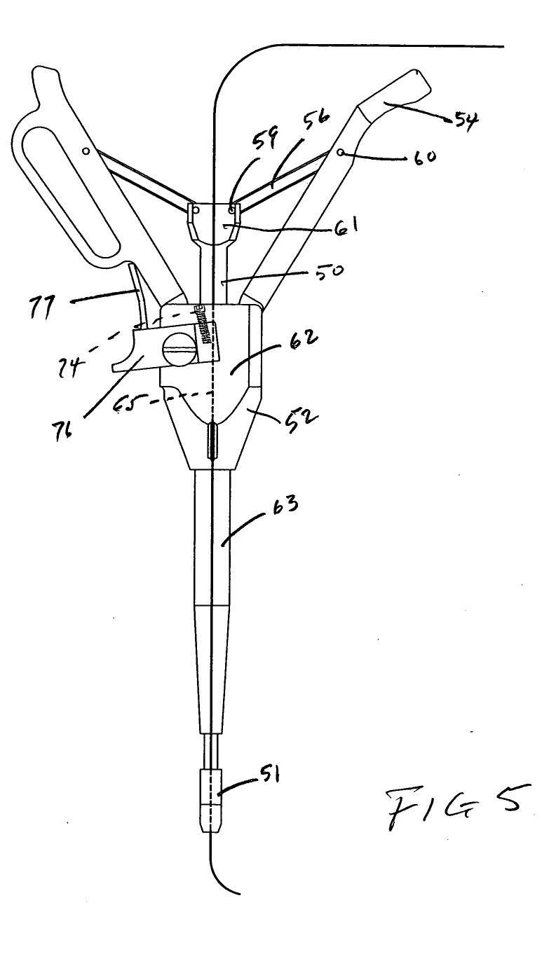

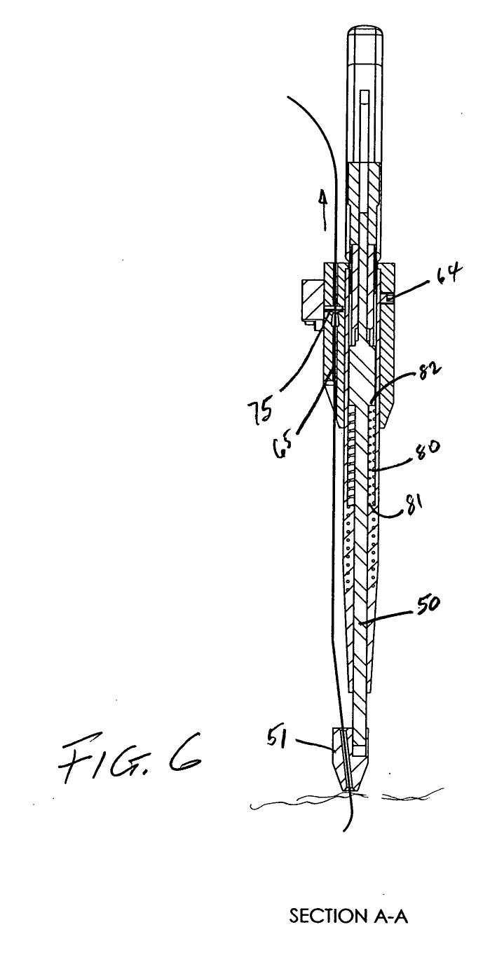

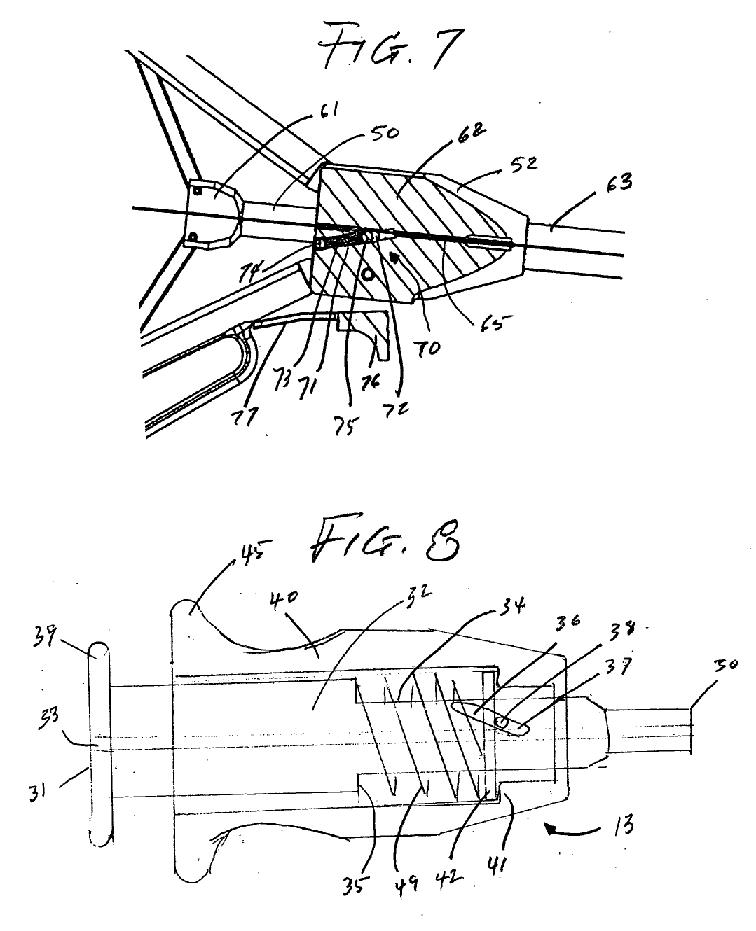

The tensioner 12, shown in FIG. 4-7, has an elongated central shaft 50 with a cable guide 51 on the distal end and a head 61 on the other end. The cable guide 51 contacts the provisional clamp 13. The cable guide 51 has an aperture therethrough with an axis parallel to the longitudinal axis of the shaft 50. The cable 11 is threaded through the cable guide and extends along the shaft to the cable chuck 52. At the proximal end of the shaft, the head 61 is connected to hand grips 53 and 54 by links 55 and 56, respectively. A pivot pin 57 connects one end of link 55 to the shaft and a pivot pin 58 connects the other end to the hand grip 53. A pivot pin 59 connects one end of link 56 to the shaft and a pivot pin 60 connects the other end to hand grip 54.

The cable chuck 52 is slidably mounted on the shaft 50 between the cable guide 51 and the head 61. The cable chuck has a body 62 and a tubular extension 63 which telescopes along the shaft 50 for directional control of the chuck and compression of a coil spring 80 surrounding the shaft. The distal ends of the hand grips 53, 54 are pivotally connected to the cable chuck body 62 by pivot pins 64. The proximal ends of the hand grips are free and spring biased to move away from each other limited by the length of the links 55, 56. The coil spring 80 has one end resting on a shoulder 81 inside the tubular extension of the chuck. The other end of the spring engages a flange 82 on the shaft. The relative movement of the shaft and the tubular extension 63 compresses the spring 80 as the hand grips move together. Release of the hand grips permits unloading of the spring and outward movement of the hand grips.

The chuck body 62 has a bore 65 through which the cable 11 passes. Within the body 62, is the clutch 70 which engages the cable 11 in the bore 65 allowing tension to be exerted by the tensioner 12, when the hand grips move toward each other, resulting in the reduction of the size of the cable loop about the bones or bone fragments.

The clutch 70 is housed in a passage 71 in the chuck body that intercepts the bore 65 at an acute angle. A clutch pin 72 is biased into the bore 65 by spring 73. The passage is closed by screw 74. Also, within the passage 71 is a transverse rod 75 resting between the clutch pin 72 and the spring 73. The rod extends through an aperture in the chuck body and is connected to a clutch arm 76. A leaf spring 77 extends between the end of the clutch arm 76 and the hand grips. As the hand grips close, the clutch arm 76 releases the rod 75 and allows the spring 73 to bias the clutch pin 72 to obstruct the cable bore 65. When the hand grips are closed and released to open, the leaf spring 77 pushes the clutch arm 76 to move the rod 75 to engage the spring 73 and remove the bias from the clutch pin 72 permitting the tensioner to move along the cable for a sequential cycle. When the clutch 70 in the tensioner 12 is released, the provisional clamp 13 maintains the cable position and prevents retrograde movement of the cable 11. The surgeon then pulls on the cable in the area between the hand grips to remove the slack from the tensioned cable and the steps are repeated until the cable is.

The components of the cable system operate most effectively when used together however, the cable, the permanent clamp, the provisional clamp and the tensioner may be employed separately, either singly or in combinations, with other conventional components.

During a surgical procedure, the skeletal bones are accessed and the desired position of the permanent clamp is selected. Depending on circumstances, the permanent clamp and the provisional clamp may be pre-installed on the cable loop. In some instances, the tensioner may also be connected to the cable. The loop is placed about the boney processes to be stabilized and the permanent clamp is placed at the desired final position. The slack is taken out of the loop which places the permanent clamp in a relatively immobile site resting on a portion of the boney processes. The increased flexibility of the cable construction permits sharper radius turns without producing slack. The provisional clamp is in contact with the permanent clamp and the tensioner is in contact with the provisional clamp. This provides a substantial span of cable oriented in a straight line which results in less distortion between the cable and the permanent clamp which, in turn, results in a tighter loop being formed. There is no deforming pressure between the cable and the permanent clamp, at this time, and this also contributes to a tighter loop.

The shaft of the tensioner is placed on the provisional clamp and, in effect, becomes immobile because of the position of the permanent clamp. As the hand grips are squeezed, the shaft remains stationary and the chuck moves away from the cable guide. Both the roller bearing in the provisional clamp and the clutch in the tensioner grip the cable and provide one-way movement of the cable in response to the movement of the chuck. When the hand grips approach each other, the squeezing pressure is released and the hand grips spring apart. This motion releases the clutch in the tensioner however, the cable is still wedged in the provisional clamp. Also, the chuck has traveled down the cable to begin a new cycle of tightening of the loop.

When the boney processes are in satisfactory stabilized position, the clutch in the tensioner is released freeing the tensioner to move along the cable. The permanent clamp is manually fixed on the cable. The provisional clamp is manually released to move along the cable and the excess cable above the permanent clamp is cut. The incision is then closed.

1. A method of using a surgical cable system comprising the steps of providing a surgical cable having a free end and a permanent clamp on the other end, looping said cable around skeletal bones, passing said free end through said permanent clamp, passing said free end through a provisional clamp, passing said free end through a manually operated tensioner, manipulating said tensioner to put tension on said cable and draw said free end through said permanent clamp and said provisional clamp reducing said loop, said provisional clamp automatically permitting passage of said cable in one direction, operating a stop in said permanent clamp when said bones are in a predetermined spatial relationship whereby said cable is crimped and the size of said loop is set and said skeletal bones are fixed in said predetermined relationship.

2. A method of claim 1 further comprising sequentially manipulating said tensioner to release said tension on said cable, moving said tensioner along said cable toward said permanent clamp and re-applying tension on said cable to draw said free end through said permanent clamp and further reduce said loop.

3. A method of claim 2 further comprising the steps of removing said tensioner from said cable.

4. A method of claim 3 further comprising the steps of manually releasing said tension in said provisional clamp and removing said provisional clamp from said cable.

5. A method of claim 1 further comprising the steps of removing said tensioer from said cable, manually releasing said tension in said provisional clamp and removing said provisional clamp from said cable.

6. A surgical cable system for forming a loop about bones and fixing the bones in a spatial relationship comprising a surgical cable with a permanent clamp on one end and a free end, said permanent clamp having a cable bore for accepting said free end of said cable, said permanent clamp including a stop in said cable bore, said stop having a first position permitting advancement of said free end through said cable bore and a second position preventing retrograde movement of said cable.

7. A surgical cable system of claim 6 comprising a lateral bore intersecting said cable bore, a mandrel ins said lateral bore adapted to expand radially and said stop slidably connected to said mandrel.

8. A surgical cable system of claim 6 comprising a multifilament cable.

9. A surgical cable system of claim 8 comprising approximately 100 to 150 filaments twisted to define a cable of great flexibility.

10. A surgical cable system of claim 6 comprising a provisional clamp, said provisional clamp having a bore therethrough for accepting said free end of said cable beyond said permanent clamp, said bore including a mechanism for contacting said cable to permit advancement of said free end and to prevent retrograde movement thereof.

11. A surgical cable system of claim 10 comprising a slot connected to said bore, a roller bearing in said slot, said roller bearing spring biased to obstruct said bore.

12. A surgical cable system of claim 10 comprising a tensioner instrument for engaging said free end of said cable beyond said provisional clamp, said instrument including a shaft with a cable guide for receiving said free end of said cable and a handle end, a cable chuck for grasping said free end of said cable between said handle end and said guide, said cable chuck slidably mounted on said shaft, said cable chuck adapted to grasp said free end of said cable as said chuck slides toward said handle end.

13. A surgical cable system of claim 12 comprising a clutch mechanism connected to said cable chuck, said clutch mechanism moving with said cable chuck to release said grasp of said cable chuck.

14. A surgical cable system of claim 12 comprising pivoting hand grips attached at said handle end to said cable chuck and said shaft whereby manipulation of said handgrips slides said cable chuck relative to said shaft.

15. A surgical cable system of claim 12 comprising said cable guide adapted to engage said mechanism on said provisional clamp and release said contact with said cable whereby said provisional clamp may be removed from said cable.

16. A surgical cable system of claim 9 comprising a tensioner instrument for engaging said free end of said cable beyond said permanent clamp, said instrument including a shaft with a guide for receiving said free end of said cable and a handle end, a cable chuck for grasping said free end of said cable beyond said guide, said cable chuck slidably mounted on said shaft, said cable chuck adapted to grasp said free end of said cable as said chuck slides toward said handle end.

17. A surgical cable system of claim 6 comprising a tensioner slidably connected to said free end of said cable, said tensioner having a shaft with a cable guide on one end and a cable chuck on the other, said cable extending through said cable guide, said chuck having a passage intersected by a spring loaded clutch, said cable passing through said passage, said clutch obstructing said passage and preventing movement of said cable.

18. A surgical cable system for forming a loop about bones and fixing the bones in a spatial relationship comprising a cable having a permanent clamp at one end and a free end, a cable bore in said permanent clamp through which said free end passes forming a loop, a stop in said cable bore permitting said free end of said cable to slide reducing said loop and preventing retrograde movement of said free end of said cable, a provisional clamp having a bore therethrough, said bore aligned with said cable bore, said bore including a mechanism contacting said free end of said cable to permit reduction of said loop and preventing retrograde movement of said cable, and a tensioner having a shaft with a guide and a handle spaced therefrom, said free end of said cable extending through said guide, a cable chuck slidably attached to said shaft near said handle, said free end of said cable extending through said cable chuck, said cable chuck grasping said cable whereby said free end of said cable is moved as said cable chuck slides toward said handle.

19. A surgical cable system of claim 18 comprising a multifilament cable.

20. A surgical cable system of claim 18 comprising said provisional clamp in contact with said permanent clamp with said bore aligned with said aperture and said provisional clamp in contact with said working end of said tensioner.

21. A surgical cable system of claim 18 comprising pivoting hand grips on said handle attached to said shaft and said cable chuck, manipulation of said handgrips sliding said cable chuck along said shaft.

22. A surgical cable system of claim 18 comprising said chuck adapted to cooperate with said mechanism to permit retrograde movement of said cable to remove said provisional clamp from said cable.

23. A surgical cable system of claim 18 comprising a clutch arm on said cable chuck, manipulation of said clutch arm releasing said grasp and permitting removal of said tensioner from said cable.
